(19) United States
(12) Patent Application Publication
(10) Pub. No.: US 2002/0177222 A1
(43) Pub. Date: Nov. 28, 2002

(54) Replication competent AAV helper functions

(76) Inventor: Sikun Li, Yueyang City (CN)

Correspondence Address: NUTTER MCCLENNEN & FISH LLP, ONE INTERNATIONAL PLACE, BOSTON, MA, 02110, US

(21) Appl. No.: 09/928,158

(22) Filed: Aug. 10, 2001

Related U.S. Application Data
(60) Provisional application No. 60/224,132, filed on Aug. 10, 2000.

(51) Int. Cl. C12N 15/861; C07H 21/04; C12N 7/00; C12Q 1/70; C07H 21/02; C12N 7/01; C12N 15/00; C12N 15/09; C12N 15/63; C12N 15/70; C12N 15/74; C12N 15/86

(52) U.S. Cl. 435/320.1; 435/456; 435/235.1; 536/23.1; 435/5

(57) ABSTRACT

The present invention provides methods and compositions for the production of high titer, wild-type-free recombinant AAV (“rAAV”) virions. The compositions of the present invention include novel nucleic acids encoding replication competent but packaging defective AAV helper functions and AAV helper function vectors. The present invention also includes host cells transfected by the claimed nucleic acids, methods of using the claimed vectors, and rAAV virions produced by such methods. The similar strategy can also be used for autonomous parvovirus virion production.

### CROSS REFERENCE TO RELATED APPLICATIONS

The present application claims the benefit, under 35 U.S.C. §119(e), of U.S. Provisional Patent Application Serial No. 60/224,132 filed Aug. 10, 2000, entitled REPLICATION COMPETENT AAV HELPER FUNCTIONS, which application is hereby incorporated herein by reference.

### BACKGROUND OF THE INVENTION

The present invention relates to methods and compositions for increasing the production of high titer stocks of recombinant AAV (rAAV) vectors. More specifically, the present invention relates to AAV helper function constructs that can mimic wild type AAV growth and provide optimized helper functions for high-efficiency rAAV production.

Parvovirinae is a subfamily of the Parvoviridae Family. Members of Parvovirinae subfamily are vertebrate viruses which include Parvovirus, Erythrovirus and Dependovirus. Essentially, these viruses fall into two groups, defective viruses which are dependent on helper virus for replication (such as adeno-associated viruses (AAV)) and autonomous, replication-competent viruses (such as Mice minute virus, B119 virus).

The particles of members of the Parvovirinae are around 18-26 nanometers(nm) in diameter. Typically, the composition of these viruses is about 50% protein and 50% DNA. The virus genomes are linear single stranded DNA in the range of 5000 nucleotides. There are three key elements in the parvovirus genome: the flanking terminal repeats, non-structural region and structural region. The terminal repeats are important cis signals for the virus replication and packaging. The non-structural region encodes the trans elements essential in regulating virus life cycle such as replication and packaging. For autonomous parvovirus, the non-structural proteins are NS1 and NS2. For AAV, the non-structural proteins are Rep proteins, which includes Rep 78, Rep 68, Rep 52 and Rep 40. The structural region encodes proteins that are components of the virion. The structural protein in AAV is called the Cap protein, which includes viral proteins (VP), in particular, VP1, VP2 and VP3. All parvoviruses are highly dependent on cellular functions for genome replication. The autonomous viruses require the cell to pass through S-phase for replication to occur. The defective parvoviruses such as AAV require host cell machinery along with helper virus (adenovirus or herpes virus) for replication.

Vectors based on parvoviruses are promising gene transfer vectors. For example, vectors based on autonomous parvovirus such as LUIII and MVM can be used for short-term gene delivery, while vectors based on dependovirus such as AAV, have the capability to direct long term transgene expression. The usefulness of the parvovirus vectors has been demonstrated in numerous in vitro and in vivo experiments. Among the parvovirus vectors, AAV vectors are the most studied prototype.

The adeno-associated virus is a nonpathogenic parvovirus with a linear, single-stranded DNA genome. Strands of both plus or minus polarity of AAV genome are packaged in separate virus particles, and the virions with either strand are equally infectious. The size of AAV virion is in the range of 18-26 nm in diameter with 50% DNA and 50% protein (Murphy, et al., (1995) “The Classification and Nomenclature of Viruses: Sixth Report of the International Committee on Taxonomy of Viruses”, Archives of Virology, (Springer-Verlag, Vienna).

Currently, there are six known primate adeno-associated virus serotypes. They are designated as AAV-1, AAV-2, AAV-3, AAV-4, AAV-5 and AAV-6. The AAV-2 is the most studied serotype. All AAVs share the basic features in the inverted terminal repeat (ITR), the rep coding region and the cap coding region. The AAV inverted terminal repeat sequences serve as the AAV replication origin, and are essential for AAV replication, packaging, integration and rescue. The ITR contains both palindromic sequences and non-palindromic sequences. The palindromic sequences can fold back on themselves to form a “T”-shaped hairpin structure. The non-palindromic sequences are designated as “D” sequences, which contain 20 nucleotides in AAV-2. D sequences are considered to be especially important for the AAV packaging (See e.g., Wang et al. (1997) J. Virol. 71: 3077-3082). The Rep region codes for at least four proteins that are required for AAV life cycles, which include Rep 78, Rep 68, Rep 52 and Rep 40. The cap region encodes AAV capsid proteins VP1, VP2 and VP3 (Murphy, et al., Supra).

AAV is a defective parvovirus, therefore requiring the presence of helper functions to replicate. In the presence of appropriate helper virus, it can be propagated as a lytic virus. Helper viruses of AAV include adenovirus, herpes simplex virus, cytomegalovirus, Epstein-Barr virus, or vaccinia virus. Without helper virus, AAV is maintained as a provirus, and integrates into host cell chromosomes, preferentially targeted to a site in chromosome 19. Integrated AAV genomes have been found to be essentially stable, persisting in tissue culture for greater than 100 passages (Muzyczka, (1992) Current Topics in Microbiol. and Immunol. 158, 97-129; Bems, “Parvoviridae and their Replication” in Fundamental Virology, 2d ed., (B. N. Fields and D. M. Knipe, eds.)).

Recombinant AAVs have many features that are of interest in the field of gene therapy. The AAV vectors are based on a defective, nonpathogenic human parvovirus that can infect both dividing and non-dividing cells. In addition, the viral genome can stably integrate within the host genome. The AAV expression vectors show little destructive cell mediated immune response. Both features facilitate long term gene transfer. The AAV vector systems have been used to express a variety of genes in many eukaryotic cells. The targets include airway epithelial cells, muscle, liver, brain, hematopoietic progenitor cells (Grimm, et al., (1999) Hum Gene Ther, 10:2445-2450).

Current methods for production of recombinant AAV (rAAV) viral stocks include three components, the rAAV vector, the AAV helper and the adenovirus helper (Samulski, et al., (1989) J. Virol, 63:3822-3828). Several other rAAV vector systems have also been designed (Li, et al., (1997) J. Virol, 71:5236-5243; Grimm, et al, (1998) Hum Gene Ther, 9:2745-60; Grimm, et al, (1999) Hum Gene Ther, 10:2445-50). Generally, in the rAAV vector, the whole coding region of AAV genome is removed to accommodate the gene of interest and only the flanking inverted terminal repeats are preserved. Adenovirus helper can be provided either as a helper virus infection or adenovirus DNA transfection. The AAV helper is used to provide AAV gene products for AAV replication and packaging.

As a typical example to produce infectious rAAV virion, a suitable producer cell line such as 293 cells is often transfected with an AAV vector plasmid along with a second plasmid harboring the AAV rep and cap genes under the control of their respective endogenous promoters or heterologous promoters. Finally, the producer cell is infected with a helper virus, such as adenovirus. Alternatively, the producer cell may be transfected with one or more vectors containing adenovirus accessory function genes. At this condition, the rAAV vector plasmid is rescued, replicated and packaged into infectious rAAV virion. Many AAV helpers have been designed to improve rAAV yields and eliminate wild type AAV contamination (Grimm, et al., (1999) Hum Gene Ther, 10:2445-2450). A common feature in all these AAV helpers is that the AAV terminal repeats are removed and only the AAV coding region is retained. One or more components for AAV production system can be established as pennanent cell lines so the infection or transfection procedures can be eliminated. It is presently possible to produce wild-type-free and adenovirus free rAAV virions, but the yields are generally low since the production often relies on tedious and inefficient transfection methods.

The major problem of the current methods of producing rAAV is the defective nature in the AAV helper function. In order to control the wild type AAV contamination in rAAV production, the terminal repeats are removed from the AAV helper constructs. This leads to a number of significant problems. First, the function of AAV terminal repeats in regulation of AAV gene expression is lost in the helper construct. Second, the copy number of AAV helper can not be increased in the rAAV replication and packaging process. At the initial stage of production process, the AAV helper copy number is low and the demands of Rep and Cap gene products are low. However, at the late stages of rAAV production, the massive replication of vector constructs creates a greater demand for AAV rep and cap gene products. Only by massive replication of the AAV helper genome can sufficient templates for transcriptions be provided to meet the demands for rep and cap gene products and, hence, high yield rAAV production. The AAV helper with the inverted terminal repeat removed will not meet the requirement of temporal optimized expression of AAV gene products. Similarly, the replicated AAV helper in SV40 or EBV vector can not meet the same requirements as it can not mimic the wild type AAV growth. Therefore, the amount of AAV helper is not synchronized to AAV replication and the temporal rep and cap expression is off. Previous studies had shown that rep gene expression has to be regulated for high rAAV yields.

From the foregoing, it will be appreciated that it would be a significant advancement in the art to provide AAV helper functions for rAAV production that can express AAV rep and cap gene in synchronization to rAAV replication and provide optimized temporal expression of the rep and cap genes. It would be a further advancement in the art to provide such helper functions that allow high efficiency production of rAAV without wild type AAV contamination.

### SUMMARY OF THE INVENTION

The similarity between the AAV and autonomous parvovirus will allow the present invention readily apply for the production of autonomous parvovirus vectors. The present invention relates to AAV helper functions for rAAV production. Provided herein are novel nucleic acid molecules that encode such AAV helper functions that will permit the AAV genome to replicate, while remaining defective for packaging. In certain embodiments, the nucleic acid molecules of the present invention comprise an AAV rep coding region, an AAV cap coding region, and at least one copy of an AAV inverted terminal repeat (ITR) or its functional equivalent that can support AAV replication. In certain preferred embodiments, AAV inverted terminal repeat is mutated so it can support AAV replication, but will be defective in AAV packaging. In certain preferred embodiments, one or more heterologous introns are also inserted into the nucleic acid molecules to make their genome sizes over the packaging capacity of AAV virions. In additional preferred embodiments, one or more heterologous DNA sequences are inserted into the non-coding region of the nucleic acid molecules. The heterologous DNA sequences can be any DNA sequences which have or do not have particular biological functions. For example, the heterologous DNA can be an intron sequence. Examples of intron sequences include, but are not limited to, α-globulin intron, β-globulin intron, collagen intron, ovalbumin intron, SV40 intron, p53 intron. The nucleic acid molecules of the present invention may be used to generate high titer stocks of rAAV without any detectable wild-type AAV contamination.

The present invention also provides AAV helper function vectors that express rep and cap gene products. Such vectors may be constructed by linking the nucleic acid molecules of the present invention with suitable control sequences that direct the replication and expression of the resulting AAV helper function vectors. An AAV helper function vector of the present invention may be a plasmid, bacteriophage, transposon, cosmid, chromosome, artificial chromosome, virus, or other suitable genetic element, and may include selectable genetic markers such as antibiotic resistance genes. Such vectors may also include one or more accessory function genes, such as the E1A, E1B, E2A, VA RNA, and E4 regions of adenovirus.

Also provided herein are host cells for producing rAAV virions. In certain embodiments, a host cell of the present invention comprises a nucleic acid encoding AAV helper functions of the present invention. Upon introduction of an AAV vector and expression of accessory functions in the host cell, rAAV virions are produced. In certain preferred embodiments, a host cell of the present invention also includes one or more accessory functions.

The present invention further provides methods of using accessory function vectors to produce rAAV virions. In certain embodiments, a method of the present invention includes the steps of (1) introducing an AAV vector into a suitable host cell; (2) introducing an AAV helper function vector of the present invention into the host cell; (3) expressing accessory functions in the host cell; and (4) culturing the host cell to produce rAAV virions. The AAV vector and AAV helper function vector can be transfected into the host cell, either sequentially or simultaneously, using well-known techniques. Accessory functions may be expressed in any of several ways, including infecting the host cell with a suitable helper virus (such as adenovirus, herpesvirus, or vaccinia virus), or by transfecting one or more accessory function vectors into the host cell. It is also well known in the art that certain cell lines, e.g., 293 cells, inherently express one or more accessory functions.

The rAAV virions produced using the present invention may be used to introduce genetic material into animals, including humans, or isolated animal cells for a variety of research and therapeutic uses. For example, rAAV virions produced using the methods of the present invention may be used to express a protein in animals to gather preclinical data or to screen for potential drug candidates. Alternatively, the rAAV virions may be used to transfer genetic material into a human to cure a genetic defect or to effect a desired treatment.

Similarly, the present invention can be modified readily for the autonomous parvovirus production. Novel nucleic acid molecules that encode such autonomous parvovirus helper functions that can replicate in the condition that autonomous parvovirus genome can replicate but are defective for packaging. In certain embodiments, the nucleic acid molecules of the present invention comprise an autonomous parvovirus non-structural protein coding region, an autonomous parvovirus structural protein coding region, and at least one copy of a parvovirus terminal repeat or its functional equivalent that can support the autonomous parvovirus replication. In certain preferred embodiments, the autonomous parvovirus terminal repeat is mutated so it can support autonomous parvovirus replication but is defective in autonomous parvovirus packaging. In certain preferred embodiments, one or more heterologous introns are inserted into the nucleic acid molecules to make their genome sizes over the packaging capacity of autonomous parvovirus virions. In additional preferred embodiments, one or more heterologous DNA sequences are inserted into the non-coding region of the nucleic acid molecules. The heterologous DNA sequences can be any DNA sequences which have or do not have particular biological functions. The nucleic acid molecules of the present invention may be used to generate high titer stocks of recombinant autonomous parvovirus without any detectable wild-type autonomous parvovirus contamination.

The present invention also provides autonomous parvovirus helper function vectors that express non-structural and structural gene products. Such vectors may be constructed by linking the nucleic acid molecules of the present invention with suitable control sequences that direct the replication and expression of the resulting autonomous parvovirus helper function vectors. An autonomous parvovirus helper function vector of the present invention may be a plasmid, bacteriophage, transposon, cosmid, chromosome, artificial chromosome, virus, or other suitable genetic element, and may include selectable genetic markers such as antibiotic resistance genes. In certain situation, a helper virus or its gene products may be used to improve the virus yield.

Also provided herein are host cells for producing recombinant autonomous parvovirus virions. In certain embodiments, a host cell of the present invention comprises a nucleic acid encoding autonomous parvovirus helper functions of the present invention. Upon introduction of a recombinant autonomous parvovirus vector and expression of accessory functions in the host cell, recombinant autonomous parvovirus virions are produced. In certain preferred embodiments, a host cell of the present invention also includes one or more accessory functions.

The present invention further provides methods of using accessory function vectors to produce recombinant autonomous parvovirus virions. In certain embodiments, a method of the present invention includes the steps of (1) introducing an autonomous parvovirus vector into a suitable host cell; (2) introducing an autonomous parvovirus helper function vector of the present invention into the host cell; (3) expressing accessory functions in the host cell; and (4) culturing the host cell to produce recombinant autonomous parvovirus virions. The recombinant autonomous parvovirus vector and autonomous parvovirus helper function vector can be transfected into the host cell, either sequentially or simultaneously, using well-known techniques. Accessory functions may be expressed in any of several ways, including infecting the host cell with a suitable helper virus (such as adenovirus, herpesvirus, or vaccinia virus), or by transfecting one or more accessory function vectors into the host cell. It is also well known in the art that certain cell lines, e.g., 293 cells, inherently express one or more accessory functions.

The recombinant autonomous parvovirus virions produced using the present invention may be used to introduce genetic material into animals, including humans, or isolated animal cells for a variety of research and therapeutic uses. For example, recombinant autonomous parvovirus virions produced using the methods of the present invention may be used to express a protein in animals to gather preclinical data or to screen for potential drug candidates. Alternatively, the recombinant autonomous parvovirus virions may be used to transfer genetic material into a human to cure a genetic defect or to effect a desired treatment.

### BRIEF DESCRIPTION OF DRAWINGS

FIG. 1 depicts the rAAV production using traditional AAV helper function. The helper plasmid does not have AAV ITRs and will not rescue and amplify in the production process.

FIG. 2 depicts the rAAV production using the AAV helper function of the present invention. The helper plasmid has AAV ITRs. It will rescue and amplify in the production process. The “*” indicates that the ITRs may be altered so they are defective for packaging. The intron insertion makes the helper plasmid too big to be encapsidated into AAV virion. This strategy will allow the high yield rAAV vector production.

FIG. 3A. depicts the cell line with the AAV helper function of the present invention. The integrated AAV helper function of the present invention will rescue and replicate in response to helper adenovirus infection. Therefore, its activity is irrelevant to its location in host genome.

FIG. 3B further depicts the cell line with the AAV helper function of the present invention. The integrated AAV helper function of the present invention will not rescue and replicate in response to helper adenovirus infection. Moreover, its activity is highly dependent on its location in host genome.

FIG. 4 depicts some examples of novel AAV helper functions of the present invention.

FIG. 5 is an illustration of components of AAV helper constructs. Key elements are identified in the figure. Note that the sizes of the plasmids are not in scale. Ay indicates mutation in ITR which affects its ability to package but still supports helper replication.

FIG. 6A is an illustration of plasmids for the establishment of stable RC AAV cell lines.

FIG. 6B is a list of cell clones obtained from RC AAV helper. The column “AAV genome amplification” indicates the clones that allow the amplification of AAV genome in the presence of adenovirus infection assayed by quantitative PCR.

FIG. 7 is a graphic characterization of AAV gene amplification in i26 and D5. RC AAV helper cell clone i26 and D5 cell line (with integrated wild type AAV genome) were infected by adenovirus at various MOI. The control group was not infected. Cells were harvested at the indicated time post adenovirus infection. Total cellular DNA was isolated and AAV DNA was quantified by Real-time PCR. The Y-axis shows amplification of AAV sequence relative to that of control group without infection by adenovirus.

FIG. 8A is a graph showing that RC AAV cell lines support rAAV production. Approximately 1.5×10⁵ D5 or i26 cells were infected with adenovirus at MOI 2, 5, 10 respectively. At 24 hrs post adenovirus infection, cells were further infected with rAAV-CAG-GFP and harvested 48 hours after rAAV infection.

FIG. 8B shows the rAAV yield and replication competent AAV contamination were determined as described in Table 2.

FIG. 9 is a graph showing how timing of adenovirus infection affects the performance of RC AAV cell line. Approximately 1.5×10⁵ i26 cells were infected with adenovirus at the MOI of 5. Cells were further infected with rAAV-CAG-GFP at the indicated time post adenovirus infection and harvested 72 hours after adenovirus infection. The rAAV was determined as described in Table 2.

FIG. 10A is a graph depicting Rep transcription in the D5 and i26 RC AAV helper cell lines.

FIG. 10B is a graph depicting Cap transcription in the D5 and i26 RC AAV helper cell lines. The D5 and i26 RC AAV helper cell lines can mimic wt AAV gene expression. Helper plasmid pAd/AAV was transfected to 293 cells. The transfected 293 cells, untransfected i26 or D5 cells were then infected with adenovirus at the MOI of 10. Cells were harvested at the indicated time points post Ad infection. Total RNAs were isolated and subjected to quantitative RT-PCR using primers specific to rep and cap transcript, respectively.

### DETAILED DESCRIPTION

All publications, patents, and patent applications cited herein are hereby incorporated by reference.

In describing the present invention, the following terms will be employed, and are defined as indicated below.

The term “gene transfer” or “gene delivery” as used herein refers to methods or systems for inserting foreign DNA into host cells. Gene transfer can result in transient expression of non-integrated transferred DNA, extrachromosomal replication and expression of transferred replicons (e.g., episomes), or integration of transferred genetic material into the genomic DNA of host cells.

The term “vector” as used herein refers to any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The term “inverted terminal repeats” or “ITRs” or “terminal repeats” as used herein refers to the art-recognized regions found at each end of a parvovirus genome which function together in cis as origins of DNA replication and as packaging signals for the viral genome.

The term “adeno-associated virus inverted terminal repeats” or “AAV ITRs” as used herein refers to the art-recognized regions found at each end of the AAV genome which function together in cis as origins of DNA replication and as packaging signals for the viral genome. AAV ITRs, together with the AAV rep coding region, provide for the efficient excision and rescue from, and integration of a nucleotide sequence interposed between two flanking ITRs into a mammalian cell genome.

Additionally, the AAV ITR may be derived from any of several AAV serotypes, including without limitation, AAV-1, AAV-2, AAV-3, AAV-4, AAV-5, AAV6, etc. As used herein, an “AAV ITR” need not have the wild-type nucleotide sequence of the above AAVs, but may be altered, e.g., by the insertion, deletion or substitution of nucleotides. In a preferred embodiment, the ITR is from the AAV-2 genome. The AAV-2 ITR have 145 nucleotides. the terminal 125 nucleotides of each ITR form palindromic hairpin (HP) structures that serve as primers for AAV DNA replication. Each ITR also contains a stretch of 20 nucleotides, designated the D sequence, which is not involved in hairpin structure formation. (See e.g., Wang et al. (1998) J. Virol. 72: 5472-5480 and Wang et al. (1997) J. Virol. 71: 3077-3082).

Regions of the inverted terminal repeats (ITR) are designated as A, B, C, A′ and D at the 5′-end of the sequences and as D, A′, B/C, C/B and A at the 3′-end of the sequences. The site between these regions is referred to as the terminal resolution site, which serves as a cleavage site in the ITRs. For example, the Rep 78 and Rep 68 possess a number of biochemical activities which include binding the viral inverted terminal repeats (ITRs), nicking at the terminal resolution site, and helicase activity. (See e.g., Kotin (1994) Hum. Gene Therap. 5:793-801 and Muzycza et al. (1992) 158: 97-129).

The term ”functional equivalent” of an AAV ITR as used herein refers to any DNA sequence that can support AAV rescue, replication, and packaging in the presence of AAV genes. It need not have the exact AAV sequence, so long as it functions as intended. It includes terminal repeat sequences from other parvoviruses which include but not limited to: LullI parvovirus (LuIII), minute virus of mice (MVM; e.g., MVMi and MVMp), hamster parvovirus (e.g., H1), feline panleukopenia virus, canine parvovirus, porcine parvovirus, latent rat virus, mink enteritis virus, human parvovirus (e.g., B19), bovine parvovirus, Aleutian mink disease parvovirus. It need not have the wild type nucleotide sequence of these parvoviruses, but may be altered, e.g., by the insertion, deletion or substitution of nucleotides.

The term “mutated AAV ITR” as used herein refers to an AAV ITR that is altered in its nucleotide sequence and loses some of its original functions or gains new functions. The alteration may be insertion, deletion or substitution of nucleotides.

The term “AAV rep coding region” refers to the art-recognized region of the AAV genome which encodes the replication proteins of the virus which are required to replicate the viral genome and to insert the viral genome into a host genome during latent infection (Muzyczka, (1992) Current Topics in Microbiol. and Immunol.; Bems, “Parvoviridae and their Replication” in Fundamental Virology, 2d ed., (B. N. Fields and D. M. Knipe, eds.). The term also includes functional homologues thereof such as the human herpesvirus 6 (HHV-6) rep gene which is also known to mediate AAV-2 DNA replication. The rep coding region, as used herein, can be derived from any viral serotype. The region need not include all of the wild-type genes but may be altered, e.g., by the insertion, deletion or substitution of nucleotides, so long as the rep genes function as intended.

The term “AAV cap coding region” as used herein refers to the art-recognized region of the AAV genome which encodes the coat proteins of the virus which are required for packaging the viral genome. The AAV cap coding region, as used herein, can be derived from any AAV serotype. The region need not include all of the wild-type cap genes but may be altered, e.g., by the insertion, deletion or substitution of nucleotides, so long as the genes provide for sufficient packaging functions when present in a host cell along with an AAV vector.

The term “AAV coding region” as used herein refers to a nucleic acid molecule that includes the two major AAV open reading frames corresponding to the AAV rep and cap coding regions. It starts from the ATG for Rep 78 to the stop codon for VP3. Thus, for purposes of the present invention, an AAV coding region does not include those sequences corresponding to the AAV p5 promoter region, and does not include the AAV ITRs. The AAV coding region, as used herein, can be derived from any AAV serotype. The region need not include all of the wild-type rep and cap genes but may be altered, e.g., by the insertion, deletion or substitution of nucleotides, so long as the genes function as intended.

The term “AAV non-coding region” as used herein refers to sequences in AAV genome between the right ITR and the ATG initiation codon for Rep 78, and the sequence between the stop codon for VP3 and the right ITR. The AAV non-coding region, as used herein, can be derived from any AAV serotype. The region may be altered, e.g., by the insertion, deletion or substitution of nucleotides, so long as it functions to support AAV life cycles.

The term “AAV vector” as used herein refers to vector derived from an adeno-associated virus serotype, including without limitation, AAV-1, AAV-2, AAV-3, AAV-4, AAV-5, AAV6, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The term “helper functions” as used herein refers to parvovirus-derived coding sequences that can be expressed to provide parvovirus gene products that, in turn, function in trans for productive parvoviurs replication.

The term “AAV helper functions” or “helpers” as used herein refer to AAV-derived coding sequences that can be expressed to provide AAV gene products that, in turn, function in trans for productive AAV replication. Thus, AAV helper functions include the rep and cap regions. The rep expression products have been shown to possess many functions, including, among others: recognition, binding and nicking of the AAV origin of DNA replication; DNA helicase activity; and modulation of transcription from AAV (or other heterologous) promoters. The cap expression products supply necessary packaging functions. AAV helper functions are used herein to complement AAV functions in trans that are missing from AAV vectors.

The term “AAV helper construct” as used herein refers generally to a nucleic acid molecule that includes nucleotide sequences which provide AAV functions. These AAV functions include the rep and cap coding regions that are replaced by a nucleotide sequence of interest in an AAV delivery vector. AAV helper constructs are commonly used to provide transient expression of AAV rep and/or cap genes to complement missing AAV functions that are necessary for lytic AAV replication; however, all previous helper constructs lack AAV ITRs and can neither replicate nor package themselves. AAV helper constructs can be in the form of a plasmid, phage, transposon, cosmid, virus, or virion. A number of other vectors have been described which encode Rep and/or Cap expression products. See, e.g., U.S. Pat. No. 5,139,941. The helper constructs of present invention include at least one copy of AAV JTR or functional equivalent to make it competent for AAV replication and rescue.

The term “accessory functions” as used herein refers to non-AAV derived viral and/or cellular functions upon which AAV is dependent for its replication (Carter, (1990) “Adeno-Associated Virus Helper Functions,” in CRC Handbook of Parvoviruses, vol. I (P. Tijssen, ed.)). Thus, the term captures DNAs, RNAs and protein that are required for AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

The term “accessory function vector” as used herein refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon, cosmid or virus that has been modified from its naturally occurring form.

The term “recombinant virus” as used herein refers to a virus that has been genetically altered, e.g., by the addition or insertion of a heterologous nucleic acid construct into the particle.

The term “AAV virion” as used herein refers to a complete virus particle, such as a to wild-type (wt) AAV virus particle (comprising a linear, single-stranded AAV nucleic acid genome associated with an AAV capsid protein coat). In this regard, single-stranded AAV nucleic acid molecules of either complementary sense, i.e., “sense” or “antisense” strands, can be packaged into any one AAV virion and both strands are equally infectious.

The term “recombinant AAV virion,” or “rAAV virion” as used herein refers to an infectious, replication-defective virus composed of an AAV protein shell encapsidating a heterologous nucleotide sequence of interest that is flanked on both sides by AAV ITRs. A rAAV virion is produced in a suitable host cell comprising an AAV vector, AAV helper functions, and accessory functions. In this manner, the host cell is rendered capable of encoding AAV polypeptides that are required for packaging the AAV vector (comprising a recombinant nucleotide sequence of interest) into infectious recombinant virion particles for subsequent gene delivery.

The term “transfection” is used herein refers to the uptake of foreign DNA by a cell. A cell has been “transfected” when exogenous DNA has been introduced inside the cell membrane. Such techniques can be used to introduce one or more exogenous DNA moieties, such as a nucleotide integration vector and other nucleic acid molecules, into suitable host cells. The term captures chemical, electrical, and viral-mediated transfection procedures.

The term “host cell” as used herein refers to, for example microorganisms, yeast cells, insect cells, and mammalian cells, that can be, or have been, used as recipients of an AAV helper construct, an AAV vector plasmid, an accessory function vector, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement to the original parent, due to natural, accidental, or deliberate mutation.

The term “cell line” as used herein refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

The term “heterologous” as it relates to nucleic acid sequences such as coding sequences and control sequences, denotes sequences which are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene).

Similarly, a cell transformed with a construct which is not normally present in the cell would be considered heterologous for purposes of this invention. Allelic variation or naturally occurring mutational events do not give rise to heterologous DNA, as used herein.

The term “heterologous DNA sequence” as used herein refers to a sequence that is foreign to the AAV nucleic acid sequence; i.e., a sequence that is not normally found in wild-type AAV nucleic acid sequences or functional equivalents thereof. As such, a heterologous nucleic acid sequence can be derived from a foreign source. It may have or does not have biological activities. For Adeno-associated virus, autonomous parvovirus nucleic acid sequences are also considered to be heterologous nucleic acid sequences.

The term “intron” as used herein refers to a nucleic acid sequence which is present in a gene that can be transcribed into RNA but is excised by RNA splicing when mRNA is produced. The introns of the present invention include all introns of any length and any class. The introns do not have to splice out 100% from all RNA transcripts. The introns of present invention can be either naturally occurring introns or synthetic introns. A “heterologous intron” here is an intron that is not presented in AAV genome.

The term “coding sequence” or a sequence which “encodes” a particular protein, as used herein refers to a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

A “nucleic acid” sequence refers to a DNA or RNA sequence. The term captures sequences that include any of the known base analogues of DNA and RNA such as, but not limited to, 4-acetylcytosine, 8-hydroxy-N6-methyladenosine, aziridinylcytosine, pseudoisocytosine, 5- (carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudo-uracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′ -methoxycarbonylmethyluracil, 5-methoxyuracil, 2-methylthio-N6-isopentenyladenine, uracil -5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, 2-thiocytosine, and 2,6-diaminopurine.

The term “promoter region” is used herein in its ordinary sense to refer to a DNA regulatory sequence to which RNA polymerase binds, initiating transcription of a downstream (3′ direction) coding sequence.

The term “AAV p5 promoter region” as used herein encompasses both promoter sequences with identity to a p5 promoter region isolated from an AAV serotype, including without limitation, AAV-1, AAV-2, AAV-3, AAV-4, AAV-5, AAV6, etc., as well as those that are substantially homologous and functionally equivalent thereto. The AAV p5 promoter directs the expression of Rep 78 and Rep 68.

The term “isolated,” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

For the purpose of describing the relative position of nucleotide sequences in a particular nucleic acid molecule throughout the instant application, such as when a particular nucleotide sequence is described as being situated “upstream,” “downstream,” “3′,” or “5′” relative to another sequence, it is to be understood that it is the position of the sequences in the “sense” or “coding” strand of a DNA molecule that is being referred to, as is conventional in the art.

The term “homology” as used herein refers to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which allow for the formation of stable duplexes between homologous regions, followed by digestion with singlestranded-specific nuclease(s), and size determination of the digested fragments. Two DNA, or two polypeptide sequences are “substantially homologous” to each other when at least about 80%, preferably at least about 90%, and most preferably at least about 95% of the nucleotides or amino acids match over a defined length of the molecules, as determined using the methods above.

The term “functional homologue” or a “functional equivalent” of a given polypeptide includes molecules derived from the native polypeptide sequence, as well as recombinantly produced or chemically synthesized polypeptides which function in a manner similar to the reference molecule to achieve a desired result. Thus, a functional homologue of AAV Rep68 or Rep78 encompasses derivatives and analogues of those polypeptides, including any single or multiple amino acid additions, substitutions and/or deletions occurring internally or at the amino or carboxy termini thereof-so long as its activity remains.

A “functional homologue” or a “functional equivalent” of a given adenoviral nucleotide region includes similar regions derived from a heterologous adenovirus serotype, nucleotide regions derived from another virus or from a cellular source, and recombinantly produced or chemically synthesized polynucleotides which function in a manner similar to the reference nucleotide region to achieve a desired result. Thus, a functional homologue of an adenoviral VA RNA gene region or an adenoviral E2A gene region encompasses to derivatives and analogues of such gene regions-including any single or multiple nucleotide base additions, substitutions and/or deletions occurring within the regions, so long as the homologue retains the ability to provide its inherent accessory function to support AAV virion production at levels detectable above background.

The term “wild-type AAV” as used herein refers to both wild-type and pseudo-wild-type AAV. Pseudo-wild-type AAV are replication-competent AAV virions produced by either homologous or non-homologous recombination between an AAV vector carrying ITRs and an AAV helper vector carrying rep and cap genes. Pseudo-wild-type AAV have nucleic acid sequences that differ from wild-type AAV sequences.

The term “parvoviruses” as used herein refers to any member of the subfamily Parvovirinae. It includes both autonomous parvovirus and dependovirus. The present invention include, but are not limited to, LuIIl parvovirus (LuIII), minute virus of mice (MVM; e.g., MVMi and MVMp), hamster parvovirus (e.g., H1), feline panleukopenia virus, canine parvovirus, porcine parvovirus, latent rat virus, mink enteritis virus, human parvovirus (e.g., B19), bovine parvovirus, Aleutian mink disease parvovirus, adeno-associated viruses (e.g., AAV-1, AAV-2).

The term “parvovirus terminal repeats” or “TR” refers to the sequences found at each end of the parvovirus genome which serve as the cis origins of DNA replication and as packaging signals for the viral genome.

The term “recombinant parvovirus virion” as used herein refers to an infectious, replication-defective virus composed of a parvovirus protein shell encapsulating a heterologous nucleotide sequence of interest that is flanked on both sides by parvovirus TRs.

The term “parvovirus virion” as used herein refers to a complete virus particle comprising a single stranded parvovirus nucleic acid genome and a parvovirus capsid coat. The term “autonomous parvoviruses” a member of subfamily Parvovirinae. It includes, but is not limited to, LuIlI parvovirus (LuIII), minute virus of mice (MVM; e.g., MVMi and MVMp), hamster parvovirus (e.g., H1), feline panleukopenia virus, canine parvovirus, porcine parvovirus, latent rat virus, mink enteritis virus, human parvovirus (e.g., B19), bovine parvovirus, Aleutian mink disease parvovirus.

The term “autonomous parvovirus coding region” as used herein refers to a nucleic acid molecule that includes the two major autonomous parvovirus open reading frames corresponding to the autonomous parvovirus non-structural and capsid protein coding regions. It starts from the ATG for NS1 to the stop codon for capsid protein gene. The region need not include all of the wild-type genes but may be altered, e.g., by the insertion, deletion or substitution of nucleotides, so long as the genes function as intended.

The term “autonomous parvovirus non-coding region” as used herein refers to sequences in autonomous parvovirus genome between the right ITR and the ATG initiation codon for non-structural protein gene (NS1), and the sequences between the stop codon for capsid gene and the right ITR. The region may be altered, e.g., by the insertion, deletion or substitution of nucleotides, so long as it functions to support autonomous parvovirus life cycles.

The term “titer” of the AAV or rAAV virions refers to the amount of AAV or rAAV virions. It was obtained by methods identical to those generally employed for determination of AAV viral titer for viral stocks prepared by conventional methods (See e.g., McLaughlin et al. (1988) J Virol. 62:1963). The particular method chosen will depend on the particular genes or other DNA carried by the virion. For example, if the virion DNA carries the GFP gene, the titer may be estimated by transducing recipient cells and measuring the frequency of expression of the GFP gene in the transductants. The genome titer is usually determined by Southern Blot or quantitative PCR. The infectious titer is determined by quantitative PCR or infectious center assay.

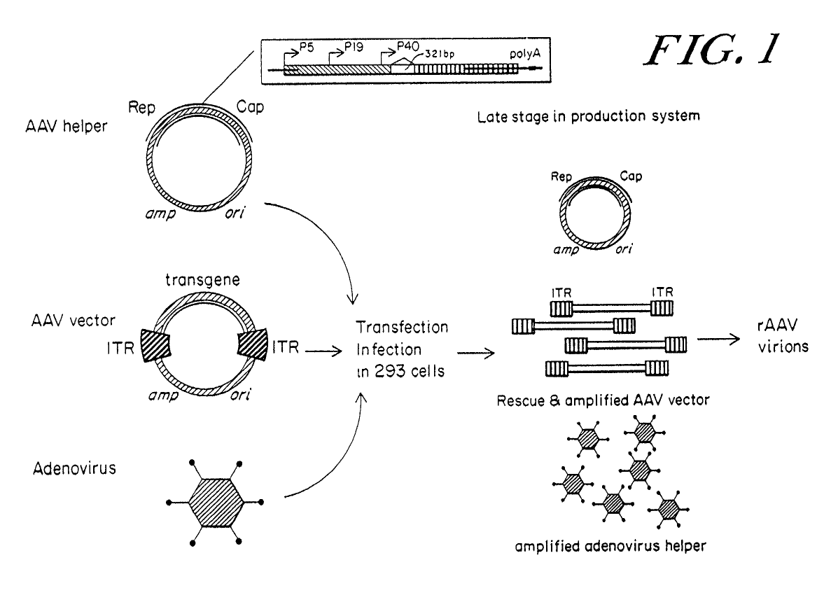

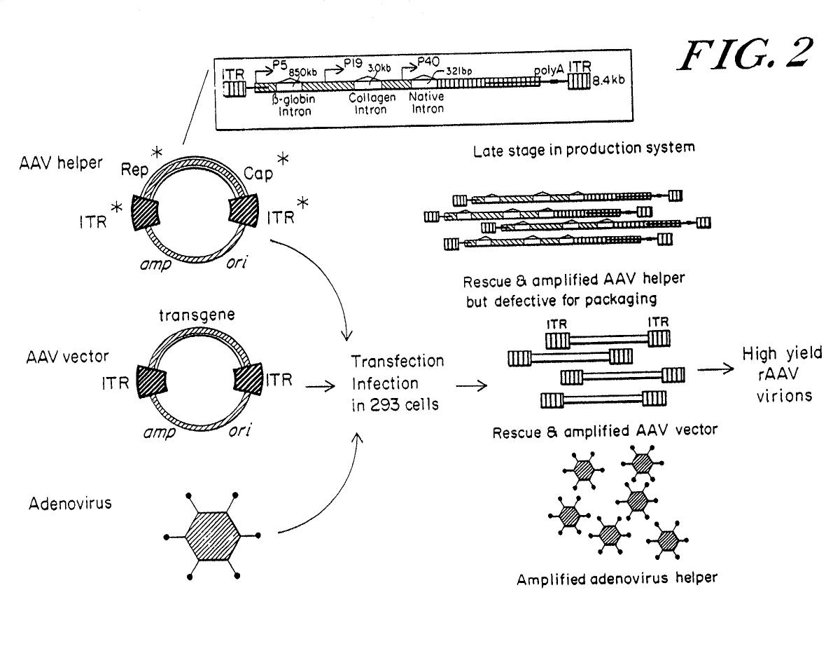

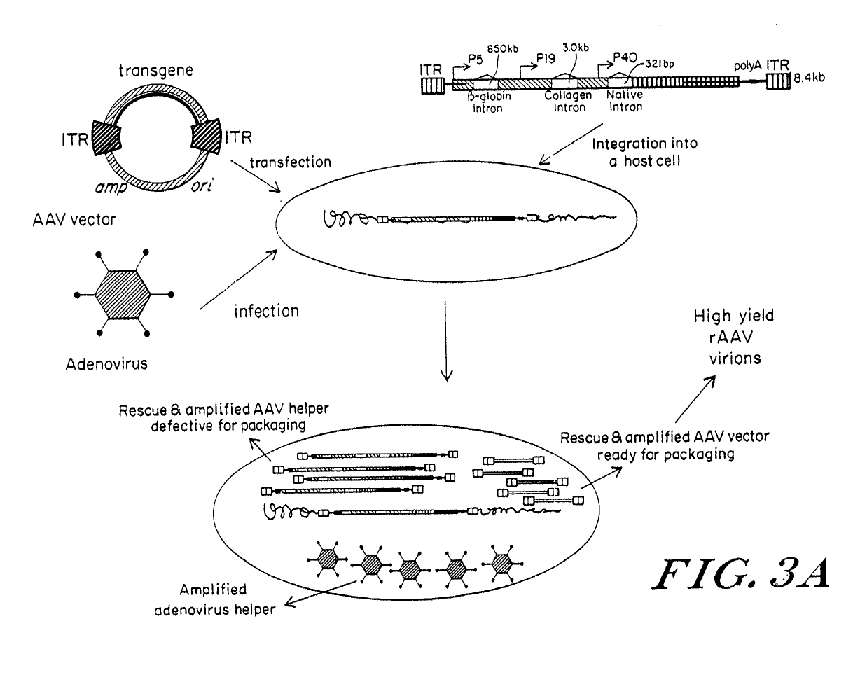

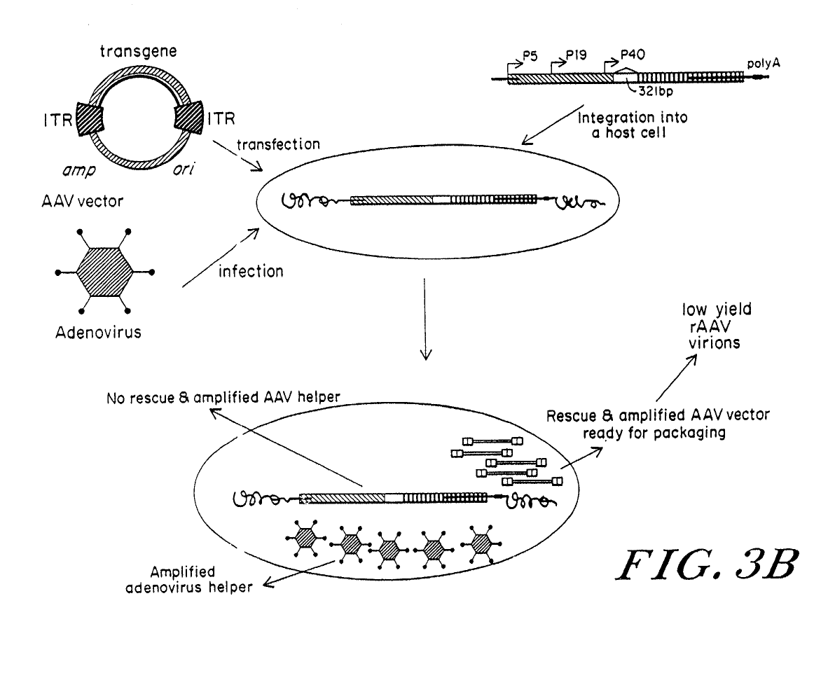

The present invention provides novel AAV helper functions for producing rAAV for introducing genetic material into animals, animal cells and human for a variety of research and therapeutic uses. Unlike previously described helper function constructs for producing rAAV which do not carry AAV ITR and can not replicate in synchronization to rAAV replication (see FIG. 1.), the AAV helper function constructs of the present invention all carry at least one copy of an AAV ITR or its functional equivalent that can support AAV replication (see FIG. 2.). The wild type AAV contamination is eliminated through using one or the combination of the following approaches: (1) including at least one copy of a mutated AAV terminal repeat or its functional equivalent that can support rAAV replication but is defective in packaging, (2) including at least one heterologous intron inserted into the AAV coding region and/or at least one heterologous DNA sequence inserted into the non coding region of the AAV helper function constructs. The insertion of the heterologous intron or it DNA sequence makes the helper construct oversize and defective in packaging. The compositions and methods of the present invention generate high titer stocks of rAAV but do not produce any detectable wild-type AAV.

The heterologous DNA used can be of any DNA sequences which may or may not have any biological activity. It may be a synthetic DNA or a coding region for another virus. In a particular case, it may be a heterologous promoter that substitutes for the p5 promoter of AAV or P4 promoter for autonomous parvovirus. When an intron is inserted to the coding region of a gene, some flanking DNA nucleotides may need to be changed so it can meet to consensus sequences for RNA spicing in mammalian cells.

In addition, the permanent cell lines with the helper function of the present invention are far superior to the cell lines based on traditional helper functions. The activity of AAV helper function of the novel cell line using the invented AAV helper functions is no longer dependent on the integration loci. Since the novel helper functions have AAV ITRs, it will rescue from the host genome and replicate along with the AAV vector sequences. Therefore, its activity is not affected by the nearby host sequences that are linked to traditionally AAV helper functions. Moreover, the helper construct comprises the AAV ITRs and can replicate in the condition that rAAV vector can replicate. This creates sufficient amount of AAV helper genome for the expression of AAV rep and cap gene. Therefore, the rAAV production cell line of the current invention is more efficient than previously described cell lines (Clark, et al., (1995) Hum Gene Ther, 6:1329-1341, Gao, et al, (1998) Hum Gene Ther, 9:2353-2362).

In one embodiment, an isolated nucleic acid molecule encoding one or more AAV helper functions for supporting rAAV virion production in an animal host cell is provided. The nucleic acid molecule comprises at least one copy of AAV ITR or its functional equivalent which can support AAV replication, AAV rep coding regions, an AAV cap coding region, AAV non-coding regions, an promoter region which includes either AAV p5 promoter or a heterologous promoter, and at least one heterologous intron (such as beta globin intron) inserted in the AAV rep and/or cap coding region.

In yet another embodiment, an isolated nucleic acid molecule encoding one or more AAV helper functions is provided. The nucleic acid molecule comprises at least one copy of an AAV ITR or its functional equivalent which can support AAV replication; an AAV rep coding region; an AAV cap coding region, AAV non-coding regions, an promoter region which includes AAV p5 promoter or a heterologous promoter, and at least one heterologous DNA sequence inserted in at least one AAV non-coding region. In further embodiments, one or multiple heterologous intron sequences were inserted in the AAV rep and/or cap coding region within the nucleic acid molecule.

In another embodiment, an isolated nucleic acid molecule encoding one or more AAV helper functions is provided. The nucleic acid molecule is replication competent but defective in packaging. It comprises at least one copy of a mutated AAV ITR or a functional equivalent to the mutated AAV ITR which can support AAV replication but is defective in supporting AAV packaging, an AAV coding region, AAV non-coding regions, and an AAV p5 promoter or a heterologous promoter. In another embodiment, it further comprises one or multiple heterologous DNA sequences inserted in the AAV non-coding region within the nucleic acid molecule. In another embodiment, it further comprises one or multiple heterologous intron sequences inserted in the AAV rep and /or cap coding region within the nucleic acid molecule. In yet another embodiment, it comprises one or multiple heterologous DNA sequences inserted in the AAV non-coding regions and one or multiple heterologous intron sequences within the nucleic acid molecule.

The intron can be inserted into any position of the AAV genome as long as it can be spliced out from the RNA transcript. It may be necessary to alter some AAV sequences to make the junctions of AAV sequences and the intron match the consensus sequence for exon -intron-exon boundary (Alberts, et al., (1994) Molecular Biology of the Cell, 3^{rd} Ed. p. 373).

The heterologous sequences inserted into the non-coding regions of AAV can be of any DNA sequence and of any length. In further embodiments, the total size of intron and heterologous DNA sequences is preferably over 5% of AAV genome size so it can reduce the packaging the AAV helper function construct.

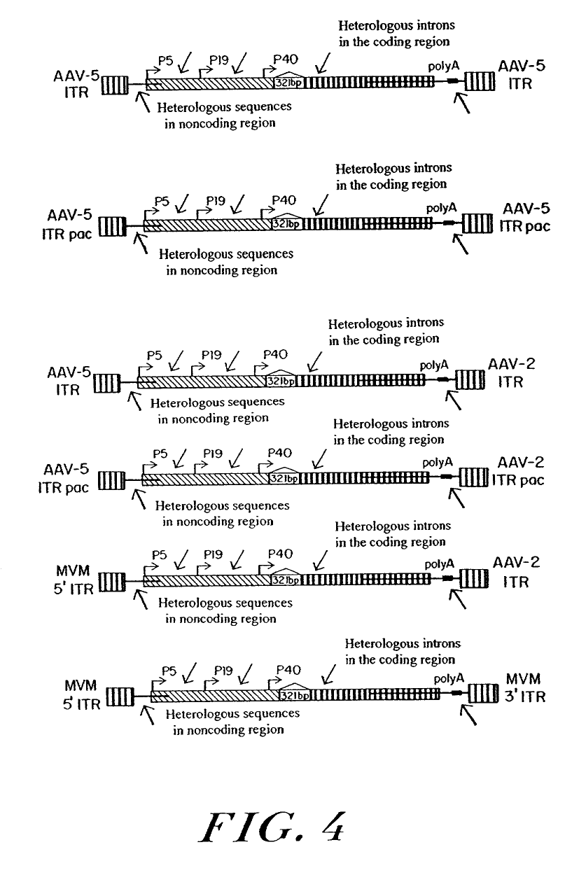

In a preferred embodiment, the nucleic acid molecule contains one or two mutated AAV ITRs comprising a deletion in the D sequence. The deletion from the D sequence can include deletion of one or more of the 20 nucleotides that constitute the D sequence beginning at the 3′ end of the ITR. In one preferred embodiment, one nucleotide of the D sequence is removed, in another preferred embodiment, 2 nucleotides, preferably 3, preferably 4, preferably 5, preferably 6, preferably 7, preferably 8, preferably 9, preferably 10, preferably 11, preferably 12, preferably 13, preferably 14, preferably 15, preferably 16, preferably 17, preferably 18, preferably 19, or preferably 20 nucleotides are deleted from the D sequence. In the most preferred embodiment, the last 15 nucleotides are deleted from the D sequence, producing an AAV ITR that only has the first 130 nucleotides of an AAV-2 ITR (SEQ ID NO: 1). The deletions to the D sequence must be such that the mutated ITR is still capable of supporting rAAV replication but not rAAV packaging. In another preferred embodiment, the nucleic acid molecule comprises only one copy of an AAV ITR at the 5′ end and no AAV ITR at the 3′ end. The 3′ end has a synthetic DNA sequence that contains AAV terminal resolution site (see FIG. 4).

The above-described nucleic acid molecules can be prepared and cloned into a suitable vector such as a plasmid or viral genome to provide an AAV helper function vector. An AAV helper function vector of the present invention can further include elements that control the replication and expression of the nucleic acid sequences that code for one or more AAV helper functions.

The AAV helper function vectors of the invention can alternatively include one or more polynucleotide homologues which replace the AAV nucleotide sequences, so long as each homologue retains the ability to provide the helper functions of the replaced AAV gene or genes. Thus, homologous nucleotide sequences can be derived from another AAV serotype or can be derived from any other suitable source.

Further, AAV helper function vectors constructed according to the invention can be in the form of a plasmid, phage, transposon, cosmid, or recombinant virus. Alternatively, the vector can be in the form of one or more linearized DNA or RNA fragments which, when associated with the appropriate control elements and enzymes, can be transcribed or expressed in a host cell to provide helper functions. All of the above-described vectors can be readily introduced into a suitable host cell using transfection techniques that are known in the art. Such transfection methods have been described, including calcium phosphate co-precipitation, direct micro-injection into cultured cells, electroporation, liposome-mediated gene transfer, lipid-mediated transfection, and nucleic acid delivery using high-velocity microprojectiles (Sambrook, et al. (1989) Molecular Cloning: A Laboratory Manual, Cold Spring Harbor Laboratories, New York).

In further embodiments, the AAV helper function vector of present invention may be present on an extrachromosomal element (such as a mini-chromosome or episome) in the host cell. Such extrachromosomal element is stably maintained in the host cell and provides the AAV helper functions essential for a productive AAV infection without the need for transfection with the AAV helper construct each time. Mammalian extrachromosomal elements are known, for example the Epstein Barr virus (EBV) based nuclear episome (Margolski, (1992), Curr. Top. Microbiol. Immun. 158:67-95) and can be readily prepared from the helper virus vector by well-known methods, for example, Giraud et al. (1994) Proc. Natl. Acad. Sci., 91:10039-10043. Preferably, the extrachromosomal element containing the helper virus vector will be present in from one to 100 copies per cell. Alternatively, the AAV helper function can be stably integrated into the chromosome of a host cell to produce a cell line which expresses the helper viral functions essential for productive AAV infection. Cell lines in which the AAV helper function is present on an extrachromosomal element or stably integrated into the chromosomal DNA can be identified and isolated by transfecting an appropriate host cell with the AAV helper vector of present invention and selecting cloned cell lines which can support the productive infection of AAV. Such cell lines can be determined by techniques that are well known in the art.

AAV helper function vectors can be engineered using conventional recombinant techniques. Particularly, nucleic acid molecules can be readily assembled in any desired order by inserting one or more accessory function nucleotide sequences into a construct, such as by ligating restriction fragments or PCR-generated products into a cloning vector using polylinker oligonucleotides or the like. The newly formed nucleic acid molecule can then be excised from the vector and placed in an appropriate expression construct using restriction enzymes or other techniques that are well known in the art.

More particularly, selected AAV nucleotide sequences or functional homologues thereof can be excised either from a viral genome or from a vector containing the same. Alternatively, selected AAV nucleotide sequences may be generated as PCR products using as a template either viral DNA or a vector containing such DNA. The nucleotide sequences are then inserted into a suitable vector either individually or linked together to provide a helper function construct using standard ligation techniques such as those described in Sambrook et al. Supra

Nucleic acid molecules comprising one or more helper functions can also be synthetically derived using a combination of solid phase direct oligonucleotide synthesis chemistry and enzymatic ligation methods that are conventional in the art. Synthetic sequences may be constructed having features such as restriction enzyme sites, and can be prepared in commercially available oligonucleotide synthesis devices such as those devices available from Applied Biosystems. Inc. (Foster City, Calif.) using the phosphoramidite method. Preferred codons for expression of the synthetic molecule in mammalian cells can also be readily synthesized. Complete nucleic acid molecules are then assembled from overlapping oligonucleotides prepared by the above methods.

The AAV helper function vectors of the present invention can be used in a variety of systems for rAAV virion production. For example, suitable host cells that have been transfected with an AAV helper function vector of the present invention are rendered capable of producing rAAV virions when co-transfected with an AAV vector and one or more accessory function vectors capable of being expressed in the cell to provide accessory functions. The AAV vector, AAV helper construct and the accessory function vector(s) can be introduced into the host cell, either simultaneously or serially, using transfection techniques described above.

AAV vectors used to produce rAAV virions for delivery of a nucleotide sequence of interest can be constructed to include one or more heterologous nucleotide sequences flanked on both ends (5′ and 3′) with functional AAV ITRs. In the practice of the invention, an AAV vector generally includes at least one AAV ITR and an appropriate promoter sequence suitably positioned relative to a heterologous nucleotide sequence, and at least one AAV ITR positioned downstream of the heterologous sequence. The 5′ and 3′ ITRs need not necessarily be identical to, or derived from, the same AAV isolate, so long as they function as intended. Such AAV vectors can be constructed using techniques well known in the art.

In the methods of the invention, accessory functions are used for rAAV production. Accessory functions may be provided by infecting the host cell with a suitable helper virus, such as adenovirus, herpesvirus, or vaccinia virus, or by transfecting the host cell with one or more accessory function vectors.

Autonomous parvovirus vector can be produced in a similar way as described for AAV vector production using replication competent autonomous parvovirus helper function. The autonomous parvovirus helper will have at least one copy of parvovirus terminal repeat to support autonomous parvovirus replication. The packaging of the helper construct is limited by using one or the combination of the following approaches: (1) including at least one copy of a mutated parvovirus terminal repeat or its functional equivalent that can support autonomous parvovirus replication but is defective in packaging, (2) including at least one heterologous DNA sequence in the autonomous parvovirus helper function constructs.

In one embodiment, an isolated nucleic acid molecule encoding one or more autonomous parvovirus helper functions for supporting recombinant autonomous parvovirus virion production in an animal host cell is provided. The nucleic acid molecule comprises at least one copy of parvovirus ITR or its functional equivalent which can support autonomous parvovirus replication, an autonomous parvovirus coding region, an autonomous parvovirus non-coding regions, an promoter region which includes either autonomous parvovirus p4 promoter or a heterologous promoter, and at least one heterologous intron (such as beta globin intron) inserted in the autonomous parvovirus coding region.

In yet another embodiment, an isolated nucleic acid molecule encoding one or more autonomous parvovirus helper functions is provided. The nucleic acid molecule comprises at least one copy of a parvovirus ITR or its functional equivalent which can support autonomous parvovirus replication; an autonomous parvovirus nonstructural protein (NS) coding region; an autonomous parvovirus cap coding region, autonomous parvovirus non-coding regions, an promoter region which includes autonomous parvovirus p4 promoter or a heterologous promoter, and at least one heterologous DNA sequence inserted in at least one autonomous parvovirus non-coding region. In further embodiments, one or multiple heterologous intron sequences were inserted in the autonomous parvovirus NS and/or cap coding region within the nucleic acid molecule.

In another embodiment, an isolated nucleic acid molecule encoding one or more autonomous parvovirus helper functions is provided. The nucleic acid molecule is replication competent but defective in packaging. It comprises at least one copy of a mutated parvovirus ITR or a functional equivalent to the mutated autonomous parvovirus ITR which can support autonomous parvovirus replication but is defective in supporting autonomous parvovirus packaging, an autonomous parvovirus coding region, autonomous parvovirus non-coding regions, and an autonomous parvovirus p4 promoter or a heterologous promoter. In another embodiment, it further comprises one or multiple heterologous DNA sequences inserted in the autonomous parvovirus non-coding region within the nucleic acid molecule. In another embodiment, it further comprises one or multiple heterologous intron sequences inserted in the autonomous parvovirus NS and /or cap coding region within the nucleic acid molecule. In yet another embodiment, it comprises one or multiple heterologous DNA sequences inserted in the autonomous parvovirus non-coding regions and one or multiple heterologous intron sequences within the nucleic acid molecule.

The intron can be inserted into any position of the autonomous parvovirus genome as long as it can be spliced out from the RNA transcript. It may be necessary to alter some autonomous parvovirus sequences to make the junctions of autonomous parvovirus sequences and the intron match the consensus sequence for exon-intron-exon boundary (Alberts et al., (1994) Molecular Biology of the Cell, 3^{rd} Ed. p. 373).

The heterologous sequences inserted into the non-coding regions of autonomous parvovirus can be of any DNA sequence and of any length. In further embodiments, the total size of intron and heterologous DNA sequences is preferably over 5% of autonomous parvovirus genome size so it can reduce the packaging the autonomous parvovirus helper function construct.

The above-described nucleic acid molecules can be prepared and cloned into a suitable vector such as a plasmid or viral genome to provide an autonomous parvovirus helper function vector. An autonomous parvovirus helper function vector of the present invention can further include elements that control the replication and expression of the nucleic acid sequences that code for one or more autonomous parvovirus helper functions.

The autonomous parvovirus helper function vectors of the invention can alternatively include one or more polynucleotide homologues which replace the autonomous parvovirus nucleotide sequences, so long as each homologue retains the ability to provide the helper functions of the replaced autonomous parvovirus gene or genes. Thus, homologous nucleotide sequences can be derived from another autonomous parvovirus serotype or can be derived from any other suitable source.

Further, autonomous parvovirus helper function vectors constructed according to the invention can be in the form of a plasmid, phage, transposon, cosmid, or recombinant virus. Alternatively, the vector can be in the form of one or more linearized DNA or RNA So fragments which, when associated with the appropriate control elements and enzymes, can be transcribed or expressed in a host cell to provide helper functions. All of the above-described vectors can be readily introduced into a suitable host cell using transfection techniques that are known in the art. Such transfection methods have been described, including calcium phosphate co-precipitation, direct micro-injection into cultured cells, electroporation, liposome-mediated gene transfer, lipid-mediated transfection, and nucleic acid delivery using high-velocity microprojectiles (Sambrook et al. (1989) Molecular Cloning: A Laboratory Manual, Cold Spring Harbor Laboratories, New York).

In further embodiments, the autonomous parvovirus helper function vector of present invention may be present on an extrachromosomal element (such as a mini-chromosome or episome) in the host cell. Such extrachromosomal element is stably maintained in the host cell and provides the autonomous parvovirus helper functions essential for a productive autonomous parvovirus infection without the need for transfection with the autonomous parvovirus helper construct each time. Mammalian extrachromosomal elements are known, for example the Epstein Barr virus (EBV) based nuclear episome (Margolski, (1992), Curr. Top. Microbiol. Immun. 158:67-95) and can be readily prepared from the helper virus vector by well-known methods, for example, Giraud et al. (1994) Proc. Natl. Acad. Sci., 91:10039-10043. Preferably, the extrachromosomal element containing the helper virus vector will be present in from one to 100 copies per cell. Alternatively, the autonomous parvovirus helper function can be stably integrated into the chromosome of a host cell to produce a cell line which expresses the helper viral functions essential for productive autonomous parvovirus infection. Cell lines in which the autonomous parvovirus helper function is present on an extrachromosomal element or stably integrated into the chromosomal DNA can be identified and isolated by transfecting an appropriate host cell with the autonomous parvovirus helper vector of present invention and selecting cloned cell lines which can support the productive infection of autonomous parvovirus. Such cell lines can be determined by techniques that are well known in the art.

Autonomous parvovirus helper function vectors can be engineered using conventional recombinant techniques. Particularly, nucleic acid molecules can be readily assembled in any desired order by inserting one or more accessory function nucleotide sequences into a construct, such as by ligating restriction fragments or PCR-generated products into a cloning vector using polylinker oligonucleotides or the like. The newly formed nucleic acid molecule can then be excised from the vector and placed in an appropriate expression construct using restriction enzymes or other techniques that are well known in the art.

More particularly, selected autonomous parvovirus nucleotide sequences or functional homologues thereof can be excised either from a viral genome or from a vector containing the same. Alternatively, selected autonomous parvovirus nucleotide sequences may be generated as PCR products using as a template either viral DNA or a vector containing such DNA. The nucleotide sequences are then inserted into a suitable vector either individually or linked together to provide a helper function construct using standard ligation techniques such as those described in Sambrook et al. Supra.

Nucleic acid molecules comprising one or more helper functions can also be synthetically derived using a combination of solid phase direct oligonucleotide synthesis chemistry and enzymatic ligation methods that are conventional in the art. Synthetic sequences may be constructed having features such as restriction enzyme sites, and can be prepared in commercially available oligonucleotide synthesis devices such as those devices available from Applied Biosystems. Inc. (Foster City, Calif.) using the phosphoramidite method. Preferred codons for expression of the synthetic molecule in mammalian cells can also be readily synthesized. Complete nucleic acid molecules are then assembled from overlapping oligonucleotides prepared by the above methods.

The autonomous parvovirus helper function vectors of the present invention can be used in a variety of systems for autonomous parvovirus virion production. For example, suitable host cells that have been transfected with an autonomous parvovirus helper function vector of the present invention are rendered capable of producing recombinant autonomous parvovirus virions when co-transfected with an autonomous parvovirus vector and one or more accessory function vectors capable of being expressed in the cell to provide accessory functions. The autonomous parvovirus vector, autonomous parvovirus helper construct and the accessory function vector(s) can be introduced into the host cell, either simultaneously or serially, using transfection techniques described above.

Autonomous parvovirus vectors used to produce recombinant autonomous parvovirus virions for delivery of a nucleotide sequence of interest can be constructed to include one or more heterologous nucleotide sequences flanked on both ends (5′ and 3′) with functional autonomous parvovirus ITRs. In the practice of the invention, an autonomous parvovirus vector generally includes at least one autonomous parvovirus ITR and an appropriate promoter sequence suitably positioned relative to a heterologous nucleotide sequence, and at least one autonomous parvovirus ITR positioned downstream of the heterologous sequence. The 5′ and 3′ ITRs need not necessarily be identical to, or derived from, the same autonomous parvovirus isolate, so long as they function as intended. Such autonomous parvovirus vectors can be constructed using techniques well known in the art.

### EXAMPLES

The following example is given to illustrate one embodiment that has been made within the scope of the present invention. It is to be understood that the following example is neither comprehensive nor exhaustive of the many types of embodiments that can be prepared in accordance with the present invention.

### Example 1

### Plasmid Construction

The plasmid piAAV-C was cloned by Xbal fragment containing AAV genome with globin intron and collagen intron from pCLR-C3k to pSub201 at XbaI. The plasmid piAAV-λ3.5k-C was cloned by replacing the BamHI/SalI fragment in piAAV-λ3.5k with the corresponding fragment containing collagen intron from pCLR-C3k. The plasmid pΔpaciAAV-C was constructed by digesting pSub201 with MscI/XhoI, recovering the large fragment, ligating with the annealed oligos DS1 5′-CCA ACT CCG-3′(SEQ ID NO: 2) and DA2 5′-AAT TCG GAG TTG G-5′(SEQ ID NO: 3), filling-in and further ligating with the filled-in XbaI fragment from piAAV-C containing globin intron and collagen intron. The plasmid pΔpaciAAV-λ3.5k-C was constructed by digesting pSub201 with MscI/Xhol, recovering the large fragment, ligating with the annealed oligos (5′-CCA ACT CCG-3′ (SEQ ID NO: 2) and 5′-AAT TCG GAG TTG G-5′(SEQ ID NO: 3)), filling-in and further ligating with the filled-in Xbal fragment from piAAV-λ3.5k-C containing globin intron and collagen intron.

### Example 2

### Comparison of AAV Production Using Traditional AAV Helper and Novel AAV Helper

Approximately seventy to eighty percent confluent 293 cells (available from ATCC, catalog number CRL-1573) were transfected with pAAV-EGFP, various helper constructs at 1:1 using calcium phosphate precipitation and infected with adenovirus at MOI 5 after transfection.

After 48 hours, the cells were harvested and centrifuged. The supernatant was removed and the remaining cell pellet was resuspended in 1 ml TBS/1% BSA. Freeze/thaw extracts were prepared by repeatedly (three times) freezing the cell suspension on dry ice and thawing at 37° C. Viral titer and wild type AAV contamination was determined according to methods published (Gao et al., (1998) Hum Gene Ther, 9:2353-62). The rAAV titer was presented as GFP forming units (GFU). The data is shown in Table 1.

TABLE 1
rAAV titre presented as GFP forming units (GFU) for different
helper functions

| | | | wtAAV | |
|---|---|---|---|---|
| | Helper | Properties | detected | |
| | size | of ITR | in dilu- | rAAV yield |
| | (kb) | (rep or pac) | tions of | GFU (units/ml) |
| pSub201 | 4.7 kb | Rep⁺ pac⁺ | 10⁶ | (3.58 ± 1.7) × 10⁴ |
| pΔpaciAAV-C | 7.7 kb | Rep⁻ pac⁺ | N.D. | (4.1 ± 0.4) × 10⁷ |
| pΔpaciAAV- | 12.0 kb | Rep⁻ pac⁺ | N.D. | (5.1 ± 0.6) × 10⁷ |
| λ3.5k-C | | | | |
| Ad/AAV | 4.7 kb | Rep⁻ pac⁻ | 10¹ | (3.3 ± 1.0) × 10⁶ |

N.D.; not detected without dilution; GFU: GFP forming unit.
Rep⁺: With replication signal.. pac⁺: with packaging signal
Rep⁻: without replication signal.. pac⁺: without packaging signal

The data showed that the new helper function can improve the yield of rAAV vector by about 1000 folds over the wild type AAV infectious clone pSub201 and about 10 to 20 folds higher than pAd/AAV. There is no replication competent AAV contamination in the vector preparations using the replication competent AAV helper.

### Example 3

### The Production of rAAV Using AAV Helper Cell Line

The Hela cell was transfected with pΔpaciAAV-C or pΔpaciAAV-λ3.5k-C by LipofectAMINE along with pcDNA3.1 at a ratio of 50:1. The transfected cells were selected under 800 μg/ml of G418. The colonies were selected and infected with adenovirus. The cell colonies that can rescue integrated pΔpaciAAV-C or pΔpaciAAV-λ3.5k-C were used for rAAV production.

To use the above cell line for rAAV production. The AAV vector plasmids were either transfected into the cell line or integrated into the host cells. Adenovirus was used for infection. The cell lysates were harvested 48 hours infection.

### Example 4

### Designs of rcAAV Helpers for rAAV Production

Various combinations of parvovirus terminal repeat or functional equivalents that can replicate under the conditions that allow AAV to replicate are shown in FIG. 4. In addition, the introns are inserted into the AAV coding region and in the non-coding region.

### Example 5

### Further Characterization of Replication Competent Helper Functions for Recombinant AAV Vector Generation

(i) Plasmid Construction

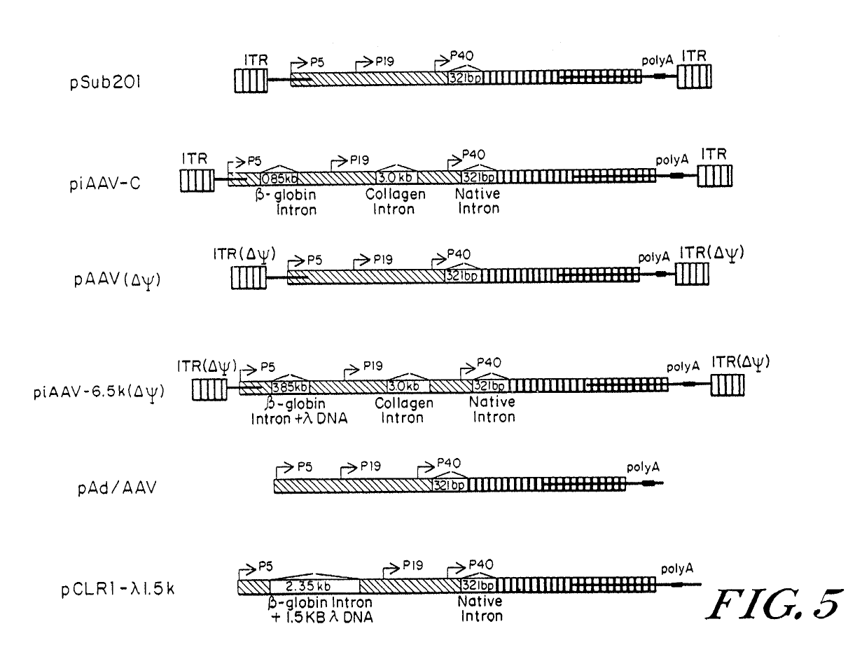

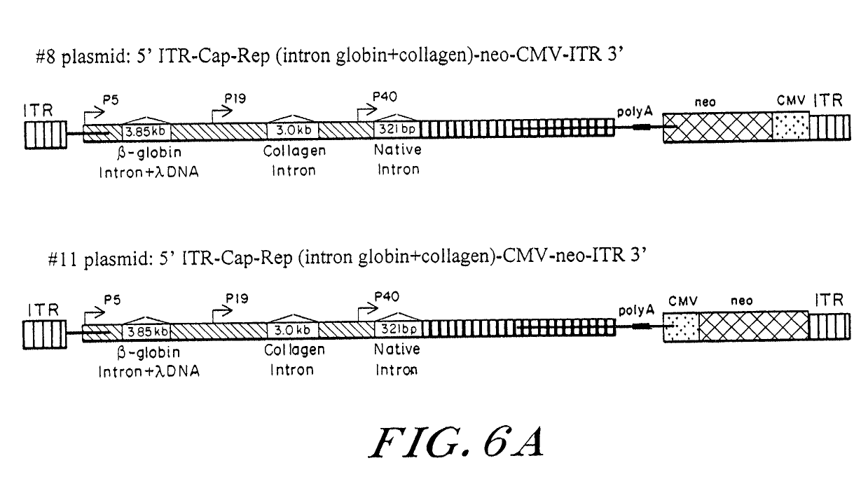

A similar approach was used to establish other helper function constructs. For example, plasmids pCLR1-λ1.5k and piAAV-C described by Cao et al. were used. These plasmids contain insertions of heterologous introns in the AAV coding region. (See Cao et al. (2000) J. Viol., 74: 11456-63). Plasmid pAAV(ΔΨ) was made by the following procedures. First, plasmid pSub201 was digested with MscI and XhoI. The fragment with hairpin sequence of ITR at both ends was then recovered and ligated to the annealed oligos of DS 1 5′ CCA ACT CCG 3′(SEQ ID NO: 2) and DA2: 5′ AAT TCG GAG TTG 3′(SEQ ID NO: 3). After ligation, the obtained fragment was filled-in with Klenow DNA polymerase and further ligated with the fragment containing the AAV genome without ITRs that was obtained from pAd/AAV by XbaI digestion and subsequent fill-in. A similar method was used to construct piAAV-6.5k (ΔΨ). Plasmid pCMV-neo was made by self-ligating the large fragment from pcDNA 3.1(−) (Invitrogen) digested with EcoR V and Smal. The plasmid pCMV-neo was digested with Sal I and then ligated to the backbone of pSub201 digested with SnaBI to obtain the pAM/CMV-neo-SV40 polyA. To make plasmids piAAV-neo(#8 and #11) for stable cell line (see FIGS. 6A and 6B), the XbaI fragment containing the AAV genome 0 including two heterologous introns (globin intron+lamda DNA sequence and collagen intron) was inserted to the Acc65I site (filled-in with Klenow) in the pAM/CMV-neo-SV40 polyA. In #8 and #11, the neo cassette is different orientation.

(ii) Replication Competent Helpers Support Wild Type Free rAAV Production in Transfection System

To test whether the replication competent AAV helpers supported AAV production, all the constructs shown in FIG. 5, were used to make rAAV-CAG-EGFP. A comparison of performance of various AAV helper constructs in triple transfection AAV production is shown in Table 2. The cells were harvested 4 days post transfection and resuspended in 1 ml 10 mM Tris-C1 (pH 8.0), freezed and thawed three times. The vector yield was determined by monitoring GFP expression in 293 cells. The wild type AAV titer was determined by serious dilution of 2 μl cell lysate and infecting 293 cells in the presence of adenovirus infection at MOI of 5. The total cellular DNA was isolated and subjected to Real-time PCR amplification of the rep sequence. At least 4-threshold cycles difference between the samples in the presence or absence of adenovirus was scored as rep replication competent.

The rAAV vector yield and contaminated wtAAV are shown in Table 2. The data demonstrates that plasmid pSub201 is an AAV infectious clone and is efficient in replication and packaging itself. The wild type AAV could be detected even after 1:106 dilution. However, the rAAV vector yield was about two to three logs lower than the other AAV helpers.

TABLE 2
A comparison of the performance of various helper constructs

| | Size % | | | wtAAV |
|---|---|---|---|---|
| | of | AAV | | titer |
| Helper | wtAAV | ITR | Vector Yield (units/ml) | (dilution) |
| pSub201 | 100% | Yes | (3.58 ± 1.71) × 10⁴ | 10⁶ |
| piAAV-C | 165% | Yes | (1.7 ± 0.4) × 10⁶ | 10⁴ |
| pAAV(Δψ) | 100% | Yes* | (6.1 ± 0.6) × 10⁷ | 10² |
| piAAV-6.5k(Δψ) | 240% | Yes* | (5.1 ± 0.6) × 10⁷ | N.D. |
| pAd/AAV | 100% | No | (3.3 ± 1.0) × 10⁶ | 10 |
| pCLR1-λ1.5k | 150% | No | (8.6 ± 2.2) × 10⁶ | N.D. |

A simple heterologous intron insertion to the AAV genome was able to increase the rAAV titer to the level of pAd/AAV because its genome was oversized to 160% of AAV genome (piAAV-C). But the replication competent AAV contamination was still quite obvious. On the other hand, replacing the ITR in pSub201 with a packaging-defective, rIo replication-permissive AAV ITR mutant pAAV(ΔΨ)) could greatly improve rAAV yield and also minimize the contamination of replication competent AAV contamination. piAAV-6.5k(ΔΨ) it carries two heterologous introns and the AAV packaging-defective ITRs. The combination of these two approaches could futher improve the rAAV yield dramatically over the intronized helper pCLRI-λ1.5k without detectable RCAAV contamination.

(iii) AAV Titer Determination

The rAAV infectious titer was determined by measuring GFP expression in 293 cells. Each green cell under fluorescence microscope represents one IU. Positive cells in the different dilutions were counted using a calibrated microscope (ocular diameter, 3.1 mm2) and then multiplied by the area of the well and the dilution of the virus. Data were represented as the average number of positive cells in a minimum of 10 fields per well.

The wild type AAV or replication competent AAV titer was determined by serial dilution of 2 μl cell lysate and infecting 293 cells in the absence or presence of adenovirus infection at moi of 5. The total cellular DNA was isolated and subjected to Real-time PCR amplification of the rep sequence. At least 4 threshold cycles difference between the samples in the presence or absence of adenovirus was scored as rep replication competent.

(iv) Establishment of Replication Competent AAV(RCAAV) Cell Lines

The two iAAV-neo helper plasmids (#8 and #11, as shown in FIG. 6A) were separately transfected to HeLa cells using LipofectAmine (GIBCO BRL) according to the manufacturer's instruction, respectively. Cells were split 48 hours post transfection and selected with 600 μg/ml Genecitin (GIBCO BRL). After selection, the majority of neomycin-resistant clones were identified to be AAV replication competent and capable of supporting rAAV production. Single cell clones were selected and expanded for further experiments. The two different constructs were similarly efficient to generate RCAAV cell lines (see FIG. 6B). These cell clones remained stable after consecutive passage.

The establishment of cell line expressing rep and cap is often a challenge and labor intensive because AAV rep proteins are highly cytotoxic when overexpressed in mammalian cells. Given AAV's biological feature of establishing its latent infection in the absence of helper virus, it was hypothesized that the replication competent AAV helper can resemble this feature of wild type AAV. Upon the presence of helper virus, the integrated AAV genome can rescue from the host chromosome. The data presented supports the idea that the integration loci should no longer remain a major concern for the performance of the helpers.

(v) Production of rAAV Using RC AAV Cell Lines

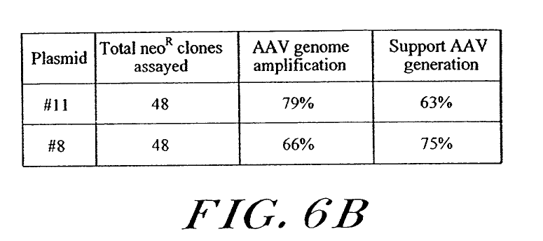

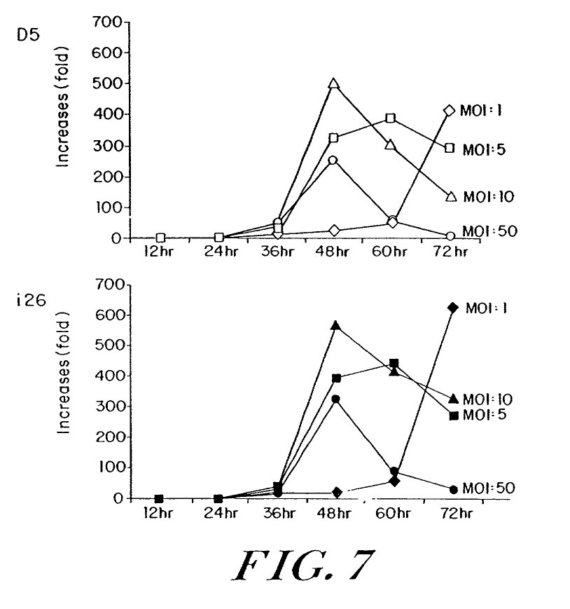

One of the rcAAV cell clone i26 was chosen to compare its helper replication to that of wild type AAV integrated cell lines Detroit 5 (D5). Both D5 and i26 were infected with helper adenovirus at various MOI. The D5 or i26 cells, at a concentration of 1.5×10⁵, were infected with adenovirus (Ad) at MOI 2, 5, 10 respectively. At 24 hrs post Ad infection, cells were further infected with rAAV-GFP at MOI of 2, and harvested 48 hours after rAAV infection. The cells were resuspended in 0.3 ml 10 mM Tris-Cl, freezed and thawed three times followed by incubation at 56° C. for 30 min to inactivate the adenovirus. The rAAV yield and replication competent AAV contamination were determined as described above. As shown in FIG. 7, D5 and i26 exhibited similar amplification curve under similar conditions.

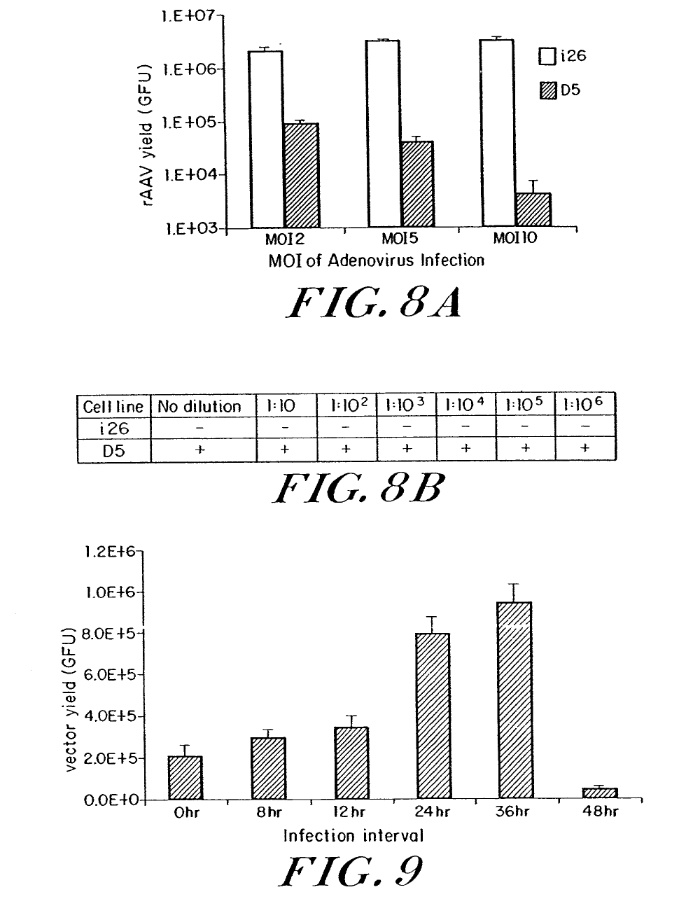

The performance of i26 in supporting rAAV production is shown in FIG. 8A. To avoid the discrepancy of transfection, the cis element was supplied by infecting i26 and D5 cell lines with rAAV-CAG-EGFP. The vectors were harvested at 72 hours post adenovirus infection. As shown in FIG. 8A, the AAV vector yield from i26 cell lines was several logs higher than that of D5 cell line at various MOI of adenovirus infection. The RCAAV contamination was well controlled in i26 cell line shown in FIG. 8B.

The performance of the RCAAV cell line was studied under a variety of conditions. One of the major factors affecting the vector yield was the adenovirus infection. For example, the time interval between rAAV infection and adenovirus infection required optimization. When rAAV and adenovirus were used to infect i26 cells simutaneously, the vectors yield was low as shown in FIG. 9. The vector yield was significantly increased when adenovirus infection took place 36 hours before rAAV infection, which was consistent to the occurrence of high level amplification of AAV genome as shown in FIG. 7. However, an insignificant number of vectors were obtained if the adenovirus infection occurred 48 hours before rAAV infection because the severe cytotoxicity derived from the long-term exposure to adenovirus infection did not allow the efficient rAAV production before the harvest time.

(vi) Assay of the Rescue and Amplification of AAV Genome Sequence in Cell Lines

Cell clones were seeded in duplicate into a 24-well plate. Each well contained approximately 5×10⁴ cells. One set was infected by adenovirus at MOI of 10 and the other was not infected to serve as control. Cells were harvested when full cytopathic effect (CPE) occurred. Total cellular DNA was isolated and subjected to Real-time PCR amplification of the rep sequence using primers (AAV-F: AAC AAG GTG GTG GAT GAG TGC (SEQ ID NO: 4); AAV-R: ACG CCC ACT GGA GCT CAG (SEQ ID NO: 5)). The quantitive PCR was carried out using PRISM/7700 Sequence Detector (PE Applied Biosystems). The reactions were performed according to the instructions of the manufacturer using the SYBR Green PCR Core Reagent Kit (PE Biosystems). The cell clones demonstrating at least 4 threshold cycles difference between the samples in the presence or absence of adenovirus were scored as positive for AAV genome amplification and selected for further experiments.

(vii) Assay of AAV Gene Expression

Helper plasmid pAd/AAV was transfected into 2×10⁶ 293 cells using LipofectAmine. The transfected 293 cells, untransfected RC AAV cell clone i26 or D5 cells were then infected with adenovirus at the MOI of 10. Cells were harvested at various time post Ad infection. Total RNAs were isolated using TRIzol LS Reagent (GIBCO BRL) according to the manufacturer' instruction. Reverse transcription reaction was performed using TaqMan Reverse Transcription Reagents (PE Biosystems ). The cDNA obtained from each sample was subjected to quantitative PCR using primers specific to rep and cap transcript, respectively, (rep-F: 5′ CGG CAA GAG GAA CAC CAT CT 3′ (SEQ ID NO: 6), rep-R: 5′ CCT CCG CGA TGT TGG TCT 3′ (SEQ ID NO: 7); cap-F: 5′ GGG CCC TGC CCA CCT 3′ (SEQ ID NO: 8), cap-R: 5′ TGT CGT TCG AGG CTC CTG A 3′ (SEQ ID NO: 9). Taqman β-actin Control Reagents (PE Biosystems ) were used as the endogenous control. All quantifications were normalized to β-actin endogenous control. Data were shown as the percentage of their maximum expression level in each cell line.

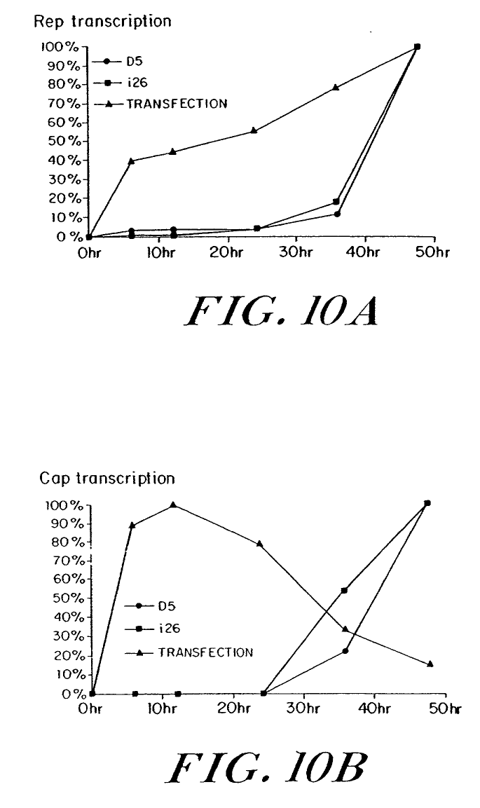

A comparison of the gene expression profile between the replication competent AAV cell line i26, D5 and transfection of pAd/AAV to 293 cells was made. Equal amount of D5, i26 cells and pAd/AAV transfected 293 cells were infected with adenovirus at MOI of 10. The total RNAs were isolated at various time points and subjected to quantitative RT-PCR. The rep transcription and cap transcription in D5 and i26 showed similar dynamic manner (see FIG. 10A). Both of the transcription remained low level until the late stage and reached their peak at 48 hours post adenovirus infection. However, the transcription in 293 cells transfected with pAd/AAV was significantly different. The rep transcription continued to go up and reached the peak at 48 hours post adenovirus infection. Its relative expression level during the early stage was much higher than that of i26 or D5. On the other hand, the cap expression quickly reached its peak at 12 hours post transfection followed by the drop of transcription, only 15% of the peak level was retained at 48 hours post infection. Such dramatic difference in expression pattern of rep and cap genes may be a reason that the transfection based AAV production system is not as efficient as that of wild type AAV growth.

Results

Replication competent AAV helper supports recombinant AAV generation in transfection based system. The strategy is based on the observation that wild type AAV growth is much more efficient than current recombinant AAV production systems. The hypothesize was that the main deficiency of rAAV production was due to the fact that the AAV ITRs are removed from the AAV helper. This deletion results in the loss of the regulating function of AAV ITRs in AAV gene expression. Moreover, it also leads to the major difference between wild type AAV growth and rAAV production in terms of the copies of rep and cap genes during the AAV growth process.

In wild type AAV growth, the early stage is limited by the number of templates available for transcription, which are sufficient to supply the necessary rep proteins for massive replication of AAV genome. In the late stage of AAV growth, when large amounts of AAV cap proteins are required for the packaging process, the existing replicating form of AAV genome allow the high level of cap expression to meet the demand of efficient encapsidation.

In contrast, in the transfection based rAAV production system, there are large amounts of AAV helper genomes available right after the transfection procedure. This leads to the oversupply of rep proteins in the early stage of rAAV production. However, in the late stage of AAV packaging, AAV templates start to decrease because of cell division. This unbalance between rep and cap may contribute to the inefficiency of the current AAV production systems. Since ITRs are critical for AAV replication, they are removed from the current AAV helper in order to prevent the generation of rcAAV contamination. The present invention, however describes a novel class of AAV helpers containing AAV ITRs. An illustration for these helper constructs is shown in FIGS. 4 and 5. These AAV helpers have demonstrated a high efficency of rAAV packaging by mimicing the wild type AAV growth characteristics. The rcAAV contamination was controlled by increasing the AAV helper genome through intron (either native or heterologous) insertion and utilizing AAV mutant ITR which are defective for encapsidation but still support AAV replication.

Collectively, these results demonstrate that the AAV helpers functions of the present invention are capable of mimicking wild type AAV growth and have a high efficiency of providing AAV transgene products for AAV packaging. These AAV helper functions carry AAV ITR and are capable of replicating themselves. The self-packaging of such helper functions were disabled by increasing the size of the helper function genome by the insertion of one or more heterologous introns and the use of encapsidation-defective mutant AAV ITRs. The data suggests that this system could not only produce rAAV vectors but also limit the extent of RCAAV contamination.

The inclusion of ITR in the packaging systems allowed the replication of AAV helper genome. However, the replication may not be the only advantage for improving the rAAV packaging. While not being limited to any mechanism, the presence of ITR in AAV helper may also restore the critical cis element for efficient regulation of AAV temporal and spatial gene expression. As demonstrated in Table 2, utilizing of rcAAV helper function could increase the vector yield up to 20 fold without any modulation of rep gene expression.

Another advantage of replication competent helper function is that the stable cell lines harboring RCAAV helper are much easier to establish. As the wild type AAV is ready to establish latent infection by the integration of its genome to the host chromosome, the RCAAV is likely to reach that efficiency. The RCAAV should also be capable of rescue from its integration loci and provide the rep and cap expression. Therefore, the performance of RCAAV cell line may be relatively independent of the integration loci.

The key to the application of RCAAV helper is to prevent the packaging of RCAAV helper function itself. As a proof of principle, the same ITR as in the vector plasmid was used. Given the homology between the helper constructs and the vector plasmids, there may remain the risk of generating fully functional ITR attached to the helper constructs by homologous recombination. The modulation of AAV packaging via heterologous intron insertion can be exploited as a safety measure to prevent RCAAV contamination. In the Examples, only two introns were inserted to the coding region of rep. Theoretically, a variety of combination of introns can potentially eliminate the possibility of RCAAV contamination derived from revertants arisen from nonhomologous recombination events.

In summary, the current invention provides AAV helper functions that include AAV ITRs or functional equivalents which allow the rescue and replication of AAV helper functions in synchronization of AAV vector sequences replication. The inclusions of AAV mutated ITR which is defective for packaging, heterologous introns in coding region, heterologous DNA sequences in non-coding region or the combination of them all help prevent the AAV helper constructs from packaging themselves.

One skilled in the art will appreciate that the methodology described in the Examples is readily applicable to autonomous parvovirus vector production, which shares a high similarity to that of rAAV production. The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. An isolated nucleic acid molecule encoding an adeno-associated virus (AAV) helper function, said nucleic acid molecule comprising:
at least one copy of an AAV inverted terminal repeat (ITR) sequence or a functional equivalent thereof that is capable of supporting AAV replication;
an AAV rep coding region; and
an AAV cap coding region.

2. The nucleic acid molecule of claim 1, further comprising at least one heterologous DNA sequence inserted at a position within said nucleic acid molecule such that the heterologous DNA sequence increases the size of the nucleic acid molecule to a size larger than the nucleic acid molecule without the heterologous sequence, wherein the increase in size prevents packaging of the AAV virus into replication competent particles.

3. The nucleic acid molecule of claim 2, wherein said heterologous DNA sequence is an intron.

4. The nucleic acid molecule of claim 1, wherein said AAV ITR is a mutated AAV ITR or a functional equivalent thereof, such that the mutated AAV ITR can support AAV replication but not AAV packaging.

5. The nucleic acid molecule of claim 4, further comprising at least one heterologous DNA sequence inserted at one or more positions within said nucleic acid molecule.

6. The nucleic acid molecule of claim 5, wherein said heterologous DNA sequence is an intron.

7. The nucleic acid molecule of claim 4, wherein said mutated AAV ITR comprises the nucleotides shown in SEQ ID NO: 1.

8. The nucleic acid molecule of claim 7, further comprising at least one heterologous DNA sequence inserted at a position within said nucleic acid molecule.

9. The nucleic acid molecule of claim 8, wherein said heterologous DNA sequence is an intron.

10. An AAV helper function vector comprising the nucleic acid molecule of claim 1, 2, 3, 4, 5, 6, 7, 8, or 9.

11. The AAV helper function vector of claim 10, further comprising at least one accessory function gene.

12. The AAV helper function vector of claim 10, wherein the vector is a plasmid.

13. A host cell comprising the nucleic acid molecule of claim 1, 2, 3, 4, 5, 6, 7, 8, or 9.

14. The host cell of claim 13 further comprising at least one accessory function gene.

15. A method of producing recombinant AAV (rAAV) virions comprising:
(a) introducing an AAV vector into a suitable host cell;
(b) introducing the AAV helper function of claim 10 into the host cell;
(c) expressing accessory functions in the host cell;
(d) culturing the host cell to produce rAAV virions; and
(e) harvesting re combinant AAV virions.

16. A recombinant AAV (rAAV) virion produced by the method of claim 15.

17. A method of producing recombinant AAV (rAAV) virions comprising:
(a) introducing an AAV vector into a host cell of claim 13;
(b) expressing accessory functions in the host cell;
(c) culturing the host cell to produce rAAV virions; and
(d) harvesting recombinant AAV virions.

18. A recombinant AAV (rAAV) virion produced by the method of claim 17.

19. An isolated nucleic acid molecule encoding an autonomous parvovirus helper function, said nucleic acid molecule comprising:
at least one copy of a parvovirus inverted terminal repeat (ITR) sequence or a functional equivalent thereof that is capable of supporting the autonomous parvovirus replication;
an autonomous parvovirus non-structural protein coding region; and
an autonomous parvovirus cap protein coding region.

20. The nucleic acid molecule of claim 19, further comprising at least one heterologous DNA sequence inserted at a position within said nucleic acid molecule such that the heterologous DNA sequence increases the size of the nucleic acid molecule to a size larger than the nucleic acid molecule without the heterologous sequence, wherein the increase in size prevents packaging of the parvovirus into replication competent particles.

21. The nucleic acid molecule of claim 20, wherein said heterologous DNA sequence is an intron.

22. The nucleic acid molecule of claim 19, wherein said autonomous parvovirus ITR is a mutated autonomous parvovirus ITR or a functional equivalent thereof, such that the mutated autonomous parvovirus ITR can support autonomous parvovirus replication but not autonomous parvovirus packaging.

23. The nucleic acid molecule of claim 22, further comprising at least one heterologous DNA sequence inserted at a position within said nucleic acid molecule.

24. The nucleic acid molecule of claim 23, wherein said heterologous DNA sequence is an intron.

25. The nucleic acid molecule of claim 19, wherein said autonomous parvovirus is MVM.

26. The nucleic acid molecule of claim 25, further comprising at least one heterologous DNA sequence inserted at a position within said nucleic acid molecule.

27. The nucleic acid molecule of claim 26, wherein said heterologous DNA sequence is an intron.

28. An autonomous parvovirus helper function vector comprising the nucleic acid molecule of claim 19, 20, 21, 22, 23, 24, 25, 26, or 27.

29. The autonomous parvovirus helper function vector of claim 28, wherein the vector is a plasmid.

30. A host cell comprising the nucleic acid molecule of claim 19, 20, 21, 22, 23, 24, 25, 26, or 27.

31. A method of producing recombinant autonomous parvovirus virions comprising:
(a) introducing an autonomous parvovirus vector into a suitable host cell;
(b) introducing the autonomous parvovirus helper function of claim 28 into the host cell;
(c) culturing the host cell to produce recombinant autonomous parvovirus virions; and
(d) harvesting recombinant autonomous parvovirus virions.

32. A recombinant autonomous parvovirus virion produced by the method of claim 31.

33. A method of producing recombinant autonomous parvovirus virions comprising:
(a) introducing an autonomous parvovirus vector into a host cell of claim 30;
(b) expressing accessory functions in the host cell;
(c) culturing the host cell to produce recombinant autonomous parvovirus virions; and
(d) harvesting recombinant autonomous parvovirus virions.

34. A recombinant AAV (rAAV) virion produced by the method of claim 17.


SEQUENCE LISTING (9 sequences)

SEQ ID NO: 1 - DNA, 130 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactcca

SEQ ID NO: 2 - DNA, 9 bp, adeno-associated virus 2
ccaactccg

SEQ ID NO: 3 - DNA, 13 bp, adeno-associated virus 2
aattcggagttgg

SEQ ID NO: 4 - DNA, 21 bp, adeno-associated virus 2
aacaaggtggtggatgagtgc

SEQ ID NO: 5 - DNA, 18 bp, adeno-associated virus 2
acgcccactggagctcag

SEQ ID NO: 6 - DNA, 20 bp, adeno-associated virus 2
cggcaagaggaacaccatct

SEQ ID NO: 7 - DNA, 18 bp, adeno-associated virus 2
cctccgcgatgttggtct

SEQ ID NO: 8 - DNA, 15 bp, adeno-associated virus 2
gggccctgcccacct

SEQ ID NO: 9 - DNA, 19 bp, adeno-associated virus 2
tgtcgttcgaggctcctga